(19) United States
(12) Patent Application Publication
(10) Pub. No.: US 2018/0256622 A1
(43) Pub. Date: Sep. 13, 2018

(54) INJECTABLE ANTIBIOTIC FORMULATIONS AND USE THEREOF

(71) Applicant: Piedmont Animal Health, LLC, Greensboro, NC (US)

(72) Inventors: Douglas I. Hepler, Greensboro, NC (US); Neil E. Paulsen, Greensboro, NC (US); Gail L. Dempsey, Greensboro, NC (US); Michael S. Daniel, Greensboro, NC (US); Tiffany G. Tomlinson, Greensboro, NC (US)

(21) Appl. No.: 15/916,057

(22) Filed: Mar. 8, 2018

Related U.S. Application Data
(60) Provisional application No. 62/598,291, filed on Dec. 13, 2017.
(60) Provisional application No. 62/563,528, filed on Sep. 26, 2017.
(60) Provisional application No. 62/469,945, filed on Mar. 10, 2017.

(51) Int. Cl. A61K 31/7052; A61K 9/00; A61K 47/10; A61K 47/32; A61K 47/14

(52) U.S. Cl. CPC A61K 31/7052; A61K 9/0019; A61K 47/14; A61K 47/32; A61K 47/10

(57) ABSTRACT

Provided herein are pharmaceutically acceptable compositions containing macrolide antibiotics, in particular azithromycin and tulathromycin. In particular, compositions containing azithromycin or tulathromycin with low toxicity, especially for administration to canines, are provided herein.

### CROSS REFERENCE TO RELATED APPLICATION(S)

This application claims the benefit of priority under 35 U.S.C. § 119(e) of U.S Ser. No. 62/598,291, filed Dec. 13, 2017, and U.S Ser. No. 62/563,528, filed Sep. 26, 2017, and U.S Ser. No. 62/469,945, filed Mar. 10, 2017, the entire content of each of which is incorporated herein by reference.

### BACKGROUND OF THE INVENTION

### FIELD OF THE INVENTION

The invention relates generally to injectable antibiotic formulations and more specifically to a formulation of an antibiotic macrolide compound with low toxicity for use in canines.

### BACKGROUND INFORMATION

The present invention is based on the seminal discovery of compositions containing an antibiotic macrolide active compound, especially azithromycin, which are formulated to be suitable for injection to animal subjects, such as canines. The formulations of the invention allow for effective treatment of infections with surprisingly lower toxicity than other available macrolide formulations. Surprisingly as well, certain macrolide formulations which have efficacy and lower toxicities in cats are not suitable for use in dogs, requiring a different strategy by species.

In the field of veterinary macrolides, azithromycin, for example, is used by veterinarians to treat a range of bacterial infections in veterinary subjects such as dogs and cats, including streptococci, staphylococci, bartonella henselae, some species of chlamydia, haemophilus spp, mycoplasma spp, borrelia burgdorferi and others. The mechanism of action of azithromycin is binding to the P site of the 50S ribosomal subunit of those microorganisms that are susceptible to it, thereby interrupting the microorganism's RNA-dependent protein synthesis. It is a semi-synthetic macrolide antibiotic derived from erythromycin. Azithromycin is a more popular choice than erythromycin in the treatment of dogs and cats because it has a longer half-life and is better absorbed by both species.

However, there are common potential side effects associated with macrolides such as azithromycin, including gastrointestinal problems like abdominal discomfort, vomiting and diarrhea. Angioedema and jaundice can also result from taking these drugs. More serious potential side effects can include cardiac arrhythmia, ventricular tachycardia and issues with renal and hepatic function.

The drug is particularly problematic for use in cats. Azithromycin in particular is cleared very slowly from feline tissue, resulting in dosage schedules that are very convenient, but an increased risk of toxicity and adverse effects in cats. A non-toxic formulation efficacious in cats has been developed (see, co-pending U.S. patent application Ser. No. 15/179,625). Surprisingly, however, the formulation did not work well in dogs. For example, the triglyceride component of the feline formulations was unexpectedly not appropriate for use in canines. The invention therefore provides compositions containing azithromycin and/or tulathromycin that are at least as potent and effective but have lower toxicity for use in canines.

### SUMMARY OF THE INVENTION

Provided herein is a composition, comprising:

(i) at least about 4% w/w and up to 15% w/w (and all concentrations in between) of a hydrated, hemi-hydrated or anhydrous form of a macrolide, such as a mectin or mycin, such as an azilide, and most especially azithromycin and tulathromycin;

(ii) at least about 1% and up to about 86% w/w of a suitable solvent (e.g., polyethylene glycol, “PEG” or polyvinylpyrrolidone); and optionally

(iii) at least about 4% w/w and up to the remaining portion of the formulation of at least one excipient (e.g., benzyl alcohol and/or ethanol).

Provided herein is a composition, comprising:

(i) at least about 4% w/w and up to 15% w/w (and all concentrations in between) of a hydrated, hemi-hydrated or anhydrous form of a macrolide, such as a mectin or mycin, such as an azilide, and most especially azithromycin and tulathromycin;

(ii) at least about 20% and up to about 60% w/w of caprylic or caprylic/capric triglycerides in combination with at least about 23% and up to about 70% w/w triacetin; and optionally

(iii) at least about 1% w/w and up to the remaining portion of the formulation of at least one excipient (e.g., Kolliphor®).

In certain aspects, the formulations are (with all concentrations in w/w of the total composition):

Azithromycin active:

15% Az, +85% PEG 300;

15% Az +4% BA +81% PEG 300;

15% Az +4% BA +0.3% HP beta-cyclodextrin +QS w/PEG 300; and

15% Az +4% BA QS w/Triacetin;

Tulathromycin Active, where BA=Benzyl Alcohol, HCl=Hydrochloric Acid, PF =Pyrogen Free, PVP=polyvinylpyrrolidone, Tula=Tulathromycin.

Tula formulations neutralized with HCl:

4% Tula, 10% BA, 6% Ethanol, 1% PVP K12PF, PEG 300;

4% Tula, 10% BA, 3% Ethanol, 1% PVP K12PF, PEG 300;

4% Tula, 10% BA, 3% Ethanol, 2% PVP K17PF, PEG 300;

4% Tula, 10% BA, 3% Ethanol, 1% PVP K17PF, PEG 300;

6% Tula, 10% BA, 5% Ethanol, 1% PVP K17PF, PEG300;

4% Tula, 10% BA, 2% Ethanol, 1% PVP K17PF, Triacetin; and

6% Tula (W_hemi), 10% BA, 1% PVP-K17 PF, 0.5% Monothioglycerol, QS with PEG300.

Tula formulations which are pH approx. 7.7 (non-aqueous) and cannot be adjusted with HCl:

4% Tula, 10% BA, 1% PVP-K17 PF, 85% Triacetin;

5% Tula, 10% BA, 1.5% PVP-K17 PF, 83.5% Triacetin;

6% Tula, 10% BA, 2% PVP-K17 PF, 82% Triacetin;

6% Tula (W_hemi), 10% BA, 2% PVP-K17 PF, 0.5% Monothioglycerol, QS with Triacetin;

6% Tula (W_hemi), 10% BA, 5% Ethanol, 1% PVP-K17 PF, 0.5% Monothioglycerol, QS with PEG300, neutralize with HCl;

4% Tula (W_hemi), 10% BA, 2% Ethanol, 1% PVP-K17 PF, 0.5% Monothioglycerol, QS with Triacetin;

6% Tula (W_or_A_anhydrous), 10% BA, 2% PVP-K17 PF, 0.5% Monothioglycerol, QS with Triacetin;

6% Tula (W_or_A_anhydrous), 10% BA, 5% Ethanol, 1% PVP-K17 PF, 0.5% Monothioglycerol, QS with PEG300;

4% Tula (W_or_A_anhydrous), 10% BA, 2% Ethanol,1% PVP-K17 PF, 0.5% Monothioglycerol, QS with Triacetin; and

4% Tula, 2% BA, 1% Kolliphor® HS15, 37% Triacetin, 56% Captex® 8000 (may be made without BA but with PVP K17PF).

Also provided herein is a method of treating an infection in an animal or a small human subject, generally a dog, with a single injection of a composition of the invention, requiring only one dose in a single injection for resolution of the infection up to 100%. No additional dosing for the infection treated should be required (although, of course, re-dosing is possible if a separate infection occurs).

In one aspect, the invention provides an injectable composition for treating or preventing an infection in a subject, in which the composition includes: a) a mono- or di-hydrate macrolide antibiotic at a concentration of between about 4 and 15% w/w of the composition; b) one or more solvents, wherein the solvent includes: 1 to 86% w/w of polyethylene glycol (PEG) or polyvinylpyrrolidone (PVP); and optionally c) an excipient.

In another aspect, the invention provides an injectable composition for treating or preventing an infection in a subject, in which the composition includes: a) a mono- or di-hydrate macrolide antibiotic at a concentration of between about 4 and 15% w/w of the composition; b) one or more solvents, wherein the solvent includes: 20 to 60% w/w caprylic or caprylic/capric triglycerides in combination with 23 to 70% w/w triacetin; and optionally c) an excipient.

The animal may be a canine including, but not limited to, a domestic dog. The method provided herein includes administering an effective amount of a composition comprising (i) a macrolide such as azithromycin or tulathromycin; (ii) an optional and suitable solvent; and optionally (iii) at least one excipient. In some aspects, the method further comprises an additional antibiotic that is co-administered with the compositions provided herein. In other aspects, the compositions are administered by injection to the canine for the treatment of an infection. The composition exhibits increased potency and efficacy, as well as reduced toxicity in canines as opposed to felines upon administration of comparable dosages to each type of mammal.

### DETAILED DESCRIPTION OF THE INVENTION

The following terms, definitions and abbreviations apply. Abbreviations used herein have their conventional meaning within the chemical and biological arts.

The term “patient” or “subject” refers to organisms, such as mammals, to be treated by the methods of the disclosure. Such organisms include, but are not limited to, horses, cats, dogs, rabbits, mice, goats, sheep, non-human primates and humans Preferably the subject is a canine such as domestic dogs. Thus, the method of the present disclosure is contemplated for use in veterinary applications. In the context of the disclosure, the term “subject” generally refers to an individual who will receive or who has received treatment described below (e.g., administration of the compounds of the disclosure, and optionally one or more additional therapeutic agents).

The term “therapeutically effective amount” means the amount of the compound or pharmaceutical composition that will elicit the biological or medical response of a patient or tissue that is being sought by the researcher, veterinarian, medical doctor or other clinician.

By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the disclosure or pharmaceutical composition to the subject in need of treatment.

The term “infection” should be understood to include invasion and/or reproduction in, a subject, of an infectious agent or organism, such as a pathogen. Pathogens should be understood to include microorganisms, for example, bacteria, viruses, fungi, and prions. As such, an infection may include any disease, disorder or symptom resulting from a viral, bacterial, or fungal infection, such as wound.

As used herein, the term “wound” refers broadly to injuries to the epithelia initiated in any one of a variety of ways (for example, pressure, inflammation, wounds induced by trauma, cuts, ulcers, burns and the like) and with varying characteristics.

A “symptom” of a wound is any morbid phenomenon or departure from the normal in structure, function, or sensation, experienced by the subject and indicative of a wound.

The term “healing” in respect to a wound refers to a process to repair a wound as by restoring the wounded tissue or epithelia to a normal state or function.

The present disclosure contemplates treating all types of wounds, including acute and chronic wounds.

The term “chronic wound” refers to a wound that exhibits impaired healing parameters interfering with the physiological sequence of events. These wounds tend to prolong and/or halt healing time course, subjecting the wounds to further complications such as recurrent infections and necrosis.

“Treatment” of a subject herein refers to both therapeutic treatment and prophylactic or preventative measures. Those in need of treatment include those already with a wound as well as those in which it is to be prevented.

The disclosure also provides pharmaceutical compositions comprising at least one active compound in an amount effective for treating a disorder, such as an infection, and a pharmaceutically acceptable vehicle or diluent. The active compound will be a macrolide antibiotic, including the mectins (including, without limitation, doximectin and abimectin) and the mycins (including, without limitation, roxithromycin, clarithromycin, tulathromycin, gamithromycin, dirithromycin, fidaxomicin, megalomicin, erythromycin and the like), potentially an azilide, and most preferably azithromycin. The active agents are most preferably hydrated; e.g., a monohydrate or dehydrate form of the molecule. The compositions of the disclosure may contain other therapeutic agents than azithromycin and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds of the disclosure may also be formulated into therapeutic compositions as natural or salt forms. Pharmaceutically acceptable non-toxic salts include the base addition salts (formed with free carboxyl or other anionic groups), which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the disclosure include amine salts formed by the protonation of an amino group with inorganic acids such as hydrochloric acid, hydrobromic acid, hydroiodic acid, sulfuric acid, phosphoric acid, and the like. Salts of the disclosure may also include amine salts formed by the protonation of an amino group with suitable organic acids, such as p-toluenesulfonic acid, acetic acid, and the like.

Additional excipients which are contemplated for use in the practice of the disclosure are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs, hydrates, and solvates of the compounds are included in the disclosure, with hydrates being particularly preferred. It should be noted that while the hydrate molecules will contribute water to the pharmaceutical composition, it is most preferred that no other water source be included.

The disclosed pharmaceutical compositions could be administered by any suitable means, for example, orally, sublingually; buccally; parenterally, such as by subcutaneous, intravenous, intramuscular, intrathecal, or intracisternal injection or infusion techniques (e.g., as sterile injectable non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. Preferably, however, the administration will be by injection or infusion, most preferably the former; e.g., by intravenous, subcutaneous or intramuscular routes of administration.

The pharmaceutical compositions for the administration of the compounds of this embodiment either alone or in combination with other agents, e.g., anti-inflammatories, analgesics, other antibiotics, anti-fungals, anti-virals and other pharmaceutically active components, although the composition is effective against infection with a hydrated macrolide, preferably an azilide, most preferably azrithromycin as the sole active agent present.

The composition may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a carrier suitable for use in an injection. In the pharmaceutical composition the active object compound is included in an amount sufficient to produce the desired effect upon the process or condition of diseases.

The pharmaceutical composition is preferably in the form of a sterile injectable solution or suspension. The composition may be formulated according to the known art using those suitable dispersing or wetting agents and suspending agents which have been mentioned herein, preferably not including water. The excipient used in the suspension is preferably polyethylene glycol (PEG) or polyvinylpyrrolidone (PVP). In embodiments, the composition is substantially non-aqueous and includes less than 2.0, 1.5, 1.0, 0.9, 0.8, 0.7, 0.6, 0.5, 0.4, 0.3, 0.2 or 0.1% w/w of water. In other embodiments, the composition includes water, wherein the water is present in an amount of up to about 2, 5, 10, 20, 30, 40, 50, 60, 70 or 80% w/w of water.

Exemplary formulations of the invention include:

(i) at least about 4% w/w and up to 15% w/w (and all concentrations in between) of a hydrated, hemi-hydrated or anhydrous form of a macrolide, such as a mectin or mycin, such as an azilide and most especially azithromycin;

(ii) at least 1% and up to about 86% of a suitable excipient (e.g., polyethylene glycol (PEG), or polyvinylpyrrolidone (PVP)); and

(iii) at least 4% w/w and up to the remaining portion of the formulation of at least one optional solvent (e.g., triacetin and/or benzyl alcohol). Where the macrolide is present in an amount of at least about 10% w/w, the solvent is optional. Where the macrolide is present in an amount of less than about 10% w/w, the solvent should be present.

In certain aspects, the formulations are (with all concentrations in w/w of the total composition):

Azithromycin active:

15% Az, +85% PEG 300;

15% Az +4% BA +81% PEG 300;

15%Az +4% BA +0.3% HP beta-cyclodextrin +QS w/PEG 300; and

15% Az +4% BA QS w/Triacetin;

Tulathromycin Active, where BA=Benzyl Alcohol, HCl=Hydrochloric Acid, PF =Pyrogen Free, PVP=polyvinylpyrrolidone, Tula=Tulathromycin.

Tula formulations neutralized with HCl:

4% Tula, 10% BA, 6% Ethanol, 1% PVP K12PF, PEG 300;

4% Tula, 10% BA, 3% Ethanol, 1% PVP K12PF, PEG 300;

4% Tula, 10% BA, 3% Ethanol, 2% PVP K17PF, PEG 300;

4% Tula, 10% BA, 3% Ethanol, 1% PVP K17PF, PEG 300;

6% Tula, 10% BA, 5% Ethanol, 1% PVP K17PF, PEG300;

4% Tula, 10% BA, 2% Ethanol, 1% PVP K17PF, Triacetin; and

6% Tula (W_hemi), 10% BA, 5% Ethanol, 1% PVP-K17 PF, 0.5% Monothioglycerol, QS with PEG300.

Tula formulations which are pH approx. 7.7 (non-aqueous) and cannot be adjusted with HCl:

4% Tula, 10% BA, 1% PVP-K17 PF, 85% Triacetin;

5% Tula, 10% BA, 1.5% PVP-K17 PF, 83.5% Triacetin;

6% Tula, 10% BA, 2% PVP-K17 PF, 82% Triacetin;

6% Tula (W_hemi), 10% BA, 2% PVP-K17 PF, 0.5% Monothioglycerol, QS with Triacetin;

6% Tula (W_hemi), 10% BA, 5% Ethanol, 1% PVP-K17 PF, 0.5% Monothioglycerol, QS with PEG300, neutralize with HCl;

4% Tula (W_hemi), 10% BA, 2% Ethanol, 1% PVP-K17 PF, 0.5% Monothioglycerol, QS with Triacetin;

6% Tula (W_or_A_anhydrous), 10% BA, 2% PVP-K17 PF, 0.5% Monothioglycerol, QS with Triacetin;

6% Tula (W_or_A_anhydrous), 10% BA, 5% Ethanol, 1% PVP-K17 PF, 0.5% Monothioglycerol, QS with PEG300; and

4% Tula (W_or_A_anhydrous), 10% BA, 2% Ethanol,1% PVP-K17 PF, 0.5% Monothioglycerol, QS with Triacetin.

In certain aspects, the excipient is polyethylene glycol, “PEG” or polyvinylpyrrolidone. In certain such embodiments, the excipient is present in an amount of up to about 50, 55, 60, 65, 66, 67, 68, 69, 70, 71, 72, 73, 74, 75, 76, 77, 78, 79, 80, 81, 82, 83, 84, 85, or 86% w/w. For example, the excipient is present in an amount of about 1 to 86%, 10 to 86%, 20 to 86%, 30 to 86%, 40 to 86%, 50 to 86%, 60 to 86%, 65 to 86%, 70 to 86%, 70 to 80% and preferably about 70 to 75.0% w/w, including about 50, 55, 60, 65, 66, 67, 68, 69, 70, 71, 72, 73, 74, 75, 76, 77, 78, 79, 80, 81, 82, 83, 84, 85, or 86% w/w.

In certain aspects, the solvent is triacetin (glyceryl triacetate or glycerol triacetate). In certain such embodiments, the solvent is present in an amount of about 23 to 70%, 30 to 60%, 40 to 55%, 34 to 45%, and preferably about 30 to 38.0% w/w, for example about 30, 31, 32, 33, 34, 35, 36, 37 or 38% w/w. Other suitable solvents may be benzyl alcohol, 2-ethoxy (2-ethoxy) ethanol, ethyl oleate, ethyl acetate, ethanol, ethyl benzoate, benzyl benzoate, 2-pyrrolidone, DMSO and 2-methyl-2pyrrolidone, 2-pyrrolidone and polyvinylpyrrolidone (e.g., PVP K17). In certain such embodiments, benzyl alcohol and/or ethanol is present in an amount of about 1 to 25%, 1 to 20%, 5 to 20%, 5 to 15%, and preferably about 5 to 15% w/w. In embodiments, benzyl alcohol is present in an amount of about 5 to 15%, 8 to 15%, 8 to 12%, or about 5, 6, 7, 8, 9, 10, 11, 12, 13, 14 or 15% w/w; and ethanol is present in an amount of about 1 to 10%, 2 to 10%, 3 to 8%, 4 to 7%, or about 1, 2, 3, 4, 5, 6, 7, 8, 9 or 10% w/w.

In another aspect, the solvent is caprylic/capric (C10 and/or C8) triglycerides or caprylic (C8) triglycerides, most preferably a C8 triglyceride. In such embodiments, the triglyceride solvent is present in an amount of about 20 to 60%, 40 to 55% and preferably about 45, 46, 47, 48, 49, 50, 51, 52, 53, 54, or 55% w/w.

In some aspects, the composition comprises a surfactant such as castor oil or hydrogenated castor oil, such as KOLLIPHOR® HS15 or RH 40 or TPGS, polysorbate (e.g., 20 and 80) or lecithen. No depot is formed in the composition of the invention. In embodiments, as KOLLIPHOR® HS15 or RH 40 or TPGS is present in an amount of about of about 0.01 to 10%, 0.05 to 10%, 0.5 to 5.0% or 0.5 to 2.5%. For example, in embodiments, the composition includes KOLLIPHOR® HS15 in an amount of up to, or about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% w/w.

The formulation can also contain other inert ingredients such as antioxidants or preservatives. Antioxidants such as a propyl gallate, BHA (butylated hydroxy anisole), BHT (butylated hydroxy toluene), MTG (monothioglycerol), tri-ethyl citrate, citric acid, TBHQ (tert-butyl hydroquinone) and the like may be added to the present formulation. The antioxidants are generally added to the formulation in amounts of from about 0.01 to about 2.0% (w/w). In certain embodiments, antioxidants are present in an amount of about 0.01 to 2.0%, 0.05 to 2.0%, 0.5 to 2.0% or 0.5 to 1.5%. For example, in embodiments, the composition includes MTG and/or citric acid in an amount of up to, or about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% w/w. In embodiments, the composition includes BHT and/or propyl galate in an amount of up to, or about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% w/w.

Preservatives such as the parabens (methylparaben and/or propylparaben) are suitably used in the formulation in amounts ranging from about 0.01 to about 2.0 w/w.

In one embodiment, the formulation of the present invention may be prepared by adding a dispersion of hydrogenated castor oil in acetylated monoglycerides, propyl dicaprylates/dicaprates or caprylic/capric triglycerides to a solution comprising the therapeutic agent. Since the formulation is intended for injection, it is desirable that it be sterilized. Surprisingly, heat sterilization may be used in crafting the formulations of the invention without adversely affecting the stability or potency of the macrolide therapeutic agent.

In the methods described herein, an appropriate dosage level will generally be about 0.01 to about 50 mg/kg, such as, for example, about 0.25 to about 15 mg/kg per day, such as about 2.0 to about 14 mg/kg per day. Within this range the dosage may be about 0.25 to 3.5 mg/kg, 0.25 to 14 mg/kg, 1.0 to 10 mg/kg, 1.5 to 10 mg/kg, 2.0 to 10 mg/kg, 2.5 to 8.0 mg/kg, 2.5 to 8 mg/kg, 2.5 to 7.0 mg/kg, 2.5 to 6.5 mg/kg, 2.5 to 6.0 mg/kg, 2.5 to 5.5 mg/kg, 2.5 to 5.0 mg/kg, 2.5 to 4.0 mg/kg, 2.5 to 3.5 mg/kg (including all intermediate dosages, such as 2.5, 2.6, 2.7, 2.8, 2.9, 3.0, 3.1, 3.2, 3.3, 3.4, 3.5, etc. mg/kg) and preferably about 3.0 mg/kg, all in a single injection form. In this form, the composition need only be administered by single injection, one time for an entire course of treatment to clinically resolve an infection up to 100% elimination. In embodiments, an infection is resolved with an efficacy greater than 90, 95, 99 or up to 100%, within a duration of less than 1, 2, 3, 4, 5, 6, 7, 8, 9, 10, 11, 12, 13, 14, 15, 16, 17, 18, 19, 20 or 21 days after a single injection form.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the patient undergoing therapy.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLE I

### Formulations

The following tulathromycin containing formulations were prepared and tested for stability. Such formulations are ideally administered to canines. They are evaluated for physical stability of the true solutions, as well as analytical results for assay, ratio of Tulathromycin A:B, and as applicable, water content or pH.

PEG 300 base formulations:

1) 6% Tula in PEG 300 with MTG; Formulation #: 1

| Ingredients | Percentages (w/w) |
|---|---|
| Tulathromycin | 6.00 |
| PEG 300 | 74.00 |
| Benzyl Alcohol | 10.00 |
| Ethanol, 190 Proof | 5.00 |
| PVP-K17 PF | 2.00 |
| Monothioglycerol (MTG) | 0.50 |
| HCl, 6.0N | 2.50 |
| TOTAL | 100.00% |

2) 6% Tula in PEG 300 with MTG and Citric Acid; Formulation #: 2

| Ingredients | Percentages (w/w) |
|---|---|
| Tulathromycin | 6.00 |
| PEG 300 | 73.00 |
| Benzyl Alcohol | 10.00 |
| Ethanol, 190 Proof | 5.00 |
| PVP-K17 PF | 2.00 |
| Monothioglycerol (MTG) | 0.50 |
| Citric Acid | 1.00 |
| HCl, 6.0N | 2.50 |
| TOTAL | 100.00% |

Triacetin base formulations:

3) 6% Tula in Triacetin and C8 triglycerides; Formulation #: 3

| Ingredients | Percentages (w/w) |
|---|---|
| Tulathromycin | 6.00 |
| Triacetin | 38.00 |
| Caprylic triglycerides | 55.00 |
| Kolliphor ® HS 15 | 1.00 |
| TOTAL | 100.00% |

4) 6% Tula in Triacetin and C8 triglycerides w/ Benzyl Alcohol &
anti-oxidants; Formulation #: 4

| Ingredients | Percentages (w/w) |
|---|---|
| Tulathromycin | 6.00 |
| Triacetin | 42.96 |
| Caprylic triglycerides | 40.00 |
| Kolliphor ® HS 15 | 1.00 |
| Benzyl Alcohol | 10.00 |
| Propyl Gallate | 0.02 |
| BHT | 0.02 |
| TOTAL | 100.00% |

5) 6% Tula in Triacetin and C8 triglycerides w/ Benzyl Alcohol,
anti-oxidants, and Benzoic Acid; Formulation #: 5

| Ingredients | Percentages (w/w) |
|---|---|
| Tulathromycin | 6.00 |
| Triacetin | 41.96 |
| Caprylic triglycerides | 40.00 |
| Kolliphor ® HS 15 | 1.00 |
| Benzyl Alcohol | 10.00 |
| Propyl Gallate | 0.02 |
| BHT | 0.02 |
| Benzoic Acid | 1.00 |
| TOTAL | 100.00% |

6) 6% Tulathromycin in Triacetin; Formulation #: 6

| Ingredients | Percentages (w/w) |
|---|---|
| Tulathromycin | 6.00 |
| Triacetin | 82.00 |
| Benzyl Alcohol | 10.00 |
| PVP-K17 PF | 2.00 |
| TOTAL | 100.00% |

7) 6% Tulathromycin in Triacetin/TEC; Formulation #: 7

| Ingredients | Percentages (w/w) |
|---|---|
| Tulathromycin | 6.00 |
| Triacetin | 50.00 |
| Triethyl Citrate (TEC) | 31.96 |
| Benzyl Alcohol | 10.00 |
| PVP-K17 PF | 2.00 |
| Propyl Gallate | 0.02 |
| BHT | 0.02 |
| TOTAL | 100.00% |

Other formulations:

8) 6% Tula; Formulation #: 8

| Ingredients | Percentages (w/w) |
|---|---|
| Tulathromycin | 6.00 |
| Water | QS |
| Propylene Glycol | 50.00 |
| Monothioglycerol (MTG) | 0.50 |
| Citric Acid | 1.00 |
| HCl, 6.0N | 1.10 |
| TOTAL | 100.00% |

9) 6% Tula in Depo-Medrol vehicle; Formulation #: 9

| Ingredients | Percentages (w/w) |
|---|---|
| Tulathromycin | 6.00 |
| PEG 3350 | 2.95 |
| Polysorbate 80 | 0.197 |
| Monobasic Sodium Phosphate | 0.69 |
| Dibasic Sodium Phosphate | 0.144 |
| Benzyl Alcohol | 0.93 |
| Water | QS |
| HCl, 6.0N | 3.00 |
| TOTAL | 100.00% |

Additional formulations:

Tulathromycin Injectable Compositions for Dogs

| Identifier | Formula (w/w) |
|---|---|
| 10 | 10% BA, 5% EtOH, 1% Citric Acid, 6% Tula, 73.0% PEG |
| | 300 pre-mixed with HCL, HCl (target pH 5.4), 0.5% MTG, |
| | 2% PVP-K17 PF |
| 11 | 10% BA, 1% Benzoic Acid, 1% Kolliphor ® HS 15, 40% |
| | C8 triglycerides, 6% Tula, 0.02% BHT, 0.02% Propyl |
| | Gallate, Triacetin |
| 12 | 6% Tula, PEG3350, Polysorbate80, Monobasic sod |
| | Phosphate, dibasic sodium phosphate, 1% BA, 86% water, |
| | HCl to pH5.58 |
| 13 | 1% Kolliphor ® HS 15, 40% C8 triglycerides, 6% Tula, |
| | 42.96% Triacetin, 10% Benzyl Alcohol, 0.02% BHT, |
| | 0.02% Propyl Gallate |
| 14 | 1% Kolliphor ® HS15, 57% Captex ® 8000, 6% Tula, |
| | 36% Triacetin |
| 15 | Water, Citric Acid, Tula, HCl, Propylene Glycol, MTG, |
| | HCl, QS with water |
| 16 | 7% Tula, 38% Triacetin, 55% Captex ® 8000 (C8), 1% |
| | Kolliphor ® HS 15 |
| 17 | 7% Tula, 46% Triacetin, 46% Captex ® 8000 (C8), 1% |
| | Kolliphor ® HS 15 |
| 18 | 6% Tula, 50% Captex ® 8000, 33% Triacetin, 10% |
| | 2-pyrrolidone, 1% Kolliphor ® HS 15 |
| 19 | 6% Tula, 50% Captex ® 8000, 34% Triacetin, 10% |
| | 2-pyrrolidone |
| 20 | 6% Tula, 50% PEG 300, 44% 2-pyrrolidone |
| 21 | 6% Tula, 50% Triacetin, 43% 2-pyrrolidone, 1% PVP-K17 |
| | PF |
| 22 | 6% Tula, 50% Triacetin, 44% 2-pyrrolidone |
| 23 | 6% Tula, 50% Captex ® 8000, 32.90% Triacetin, 10% |
| | 2-pyrrolidone, 1% Kolliphor ® HS 15, 0.05% BHT, 0.05% |
| | Propyl Gallate |
| 24 | 6% Tula, 50% Captex ® 8000, 33.90% Triacetin, 10% |
| | 2-pyrrolidone, 0.05% BHT, 0.05% Propyl Gallate |

### EXAMPLE II

### Efficacy and Safety

This study will be to evaluate the effectiveness and field safety of a single injection of a tulathromycin formulation for the treatment of skin and soft tissue infections (abscesses) in dogs.

At least twenty dogs will be enrolled in the study. All dogs will be treated with the investigational veterinary product (IVP), and all be will be included in the efficacy evaluation.

Dogss enrolled in the study will be presented to the clinic with skin and soft tissue infections. On Day 0, a physical examination will be conducted, which includes assignment of a wound clinical score based on swelling, pain, and exudate. For inclusion in the study, the wound clinical score will be a minimum of 5, with a minimum exudate score of 2. A swab will be obtained from the wound (following lancing for closed abscesses) and shipped to each investigator's preferred contract laboratory for bacterial culture. Wound management procedures will be allowed after swab collections, but the only permissible cleaning agents were saline or tap water. Blood and serum samples will be collected, and hematology and serum chemistry analyses will be conducted in-clinic.

The dogs will be dosed via subcutaneous injection with about 3 mg/kg of a composition as described in Example I, preferably formulations 1, 2, 10 and/or 23.

Observations will be made hourly for the first 4 hours postinjection, and an injection site observation and temperature will be obtained at 4 hours post-injection. At approximately 24 hours post-injection, another temperature and injection site evaluation will be conducted.

At the interim study visit on Day 7 (±2), a wound clinical score will be assigned and the injection site will be examined for any abnormalities. At the final visit on Day 14 (±2), a physical examination will be conducted, a wound clinical score will be assigned, and the injection site will be evaluated for any abnormalities. In addition, blood and serum samples will be collected for a final hematology and serum chemistry analysis.

A successful case will be defined as a dog with a concluding wound score of 1 for at least two of the three variables, and an improvement of at least 1 or a score of 1 in the third variable.

Based on wound clinical scores, we expect all dogs in the effectiveness analysis to be considered successful cases, resulting in an efficacy of 100%.

### EXAMPLE III

### Toxicity Evaluation

Dose levels of control, 3 mg/kg (Day 0) and 15 mg/kg (Day 0) of tulathromycin active prepared in the formulations of Example I (formulations 1, 2, 10 and 23) will be evaluated for adverse reactions in canines.

Dogs will be dosed as described in the Examples above via subcutaneous (SQ) injection into the right dorsoscapular area. Injection sites will be evaluated once in acclimation, at four hours post-dosing, and once daily from Days 1 to 7. Rectal temperatures will be taken at four hours post-dosing, once daily post-dosing, then discontinued at three days post-dosing if rectal temperatures remained within test facility reference ranges. Blood will be collected for clinical pathology (hematology and clinical chemistry) from all dogs on Study Days -7, 3, and 7. Standard six-lead and rhythm strip electrocardiographs (ECGs) will be obtained from each dog once during acclimation, on Day 0 at two hours post-dosing, on Day 1 at 24 hours post-dosing, and then on Day 7.

In conclusion, tulathromycin 7% injection, when administered SQ in dogs at 1 and 5 times the proposed label dose versus a placebo control is expected to not be associated with any clinically or toxicologically relevant effects on clinical chemistry, hematology, ECG, rectal temperature, or food consumption.

Although the objects of the disclosure have been described with reference to the above example, it will be understood that modifications and variations are encompassed within the spirit and scope of the disclosure. Accordingly, the disclosure is limited only by the following claims.

What is claimed is:

1. An injectable composition for treating or preventing an infection in a subject, the composition comprising:
a) a mono- or di-hydrate macrolide antibiotic at a concentration of between about 4 and 15% w/w of the composition;
b) one or more solvents, wherein the solvent comprises:
(i) 1 to 86% w/w of polyethylene glycol (PEG) or polyvinylpyrrolidone (PVP); or
(ii) 20 to 60% w/w caprylic or caprylic/capric triglycerides in combination with 23 to 70% w/w triacetin; and optionally
c) an excipient.

2. The composition of claim 1, wherein the antibiotic is at a concentration of about 4, 5, 6 or 7% w/w of the composition.

3. The composition of claim 1, wherein the antibiotic is azithromycin or tulathromycin.

4. The composition of claim 1, wherein the solvent comprises PEG at a concentration of between about 70 to 86% w/w and PVP at a concentration of between about 1 to 3% w/w.

5. The composition of claim 4, wherein the solvent comprises PEG at a concentration of about 73 or 74% w/w and PVP at a concentration of about 2% w/w.

6. The composition of claim 4, wherein the excipient is benzyl alcohol, ethanol, or a combination thereof.

7. The composition of claim 1, wherein caprylic or caprylic/capric triglycerides are present in a concentration of about 50 to 55% w/w, and triacetin is present in a concentration of about 30 to 38% w/w.

8. The composition of claim 7, wherein the excipient is KOLLIPHOR® HS15.

9. The composition of claim 1, wherein the composition provides antibiotic sufficient to resolve an infection without toxicity to the subject within about 7 days of administration to a subject.

10. The composition of claim 2, wherein azithromycin or tulathromycin is present in a dose of about 2.0, 2.5, 3.0, 3.5, 4.0, 4.5, 5.0, 5.5, 6.0, 6.5 or 7.0 mg/kg.

11. The composition of claim 1, wherein the composition is stable at room temperature for at least 6 months.

12. The composition of claim 11, wherein the composition is stable at room temperature for at least 12 months.

13. A method of treating or preventing an infection in a subject, comprising administering to the subject an effective amount of a composition of claim 1 by injection, thereby treating or preventing the infection.

14. The method of claim 13, further comprising administering an additional antibiotic.

15. The method of claim 13, further comprising administering a therapeutic agent in combination with the composition of claim 1, wherein the therapeutic agent is selected from an anti-inflammatory, analgesic, anti-fungal or anti-viral.

16. The method of claim 14, wherein the subject is a canine.

17. The method of claim 13, wherein infection is resolved following a single administration.

18. The method of claim 13, wherein the infection is a microbial infection.

19. The method of claim 18, wherein the microbe is a virus, bacteria or fungi.

20. The method of claim 13, wherein the infection is resolved without toxicity to the subject within about 7 days of treatment.

24. A kit for treating an infection in a subject comprising:
a) the composition of claim 1 prepackaged in a sterile syringe; and
b) instructions for administering the composition to the subject.
